(19) United States
(12) Patent Application Publication
(10) Pub. No.: US 2003/0230312 A1
(43) Pub. Date: Dec. 18, 2003

(54) Adhesive skin protective patch for forefoot

(76) Inventor: Renata Irina Dumitrascu, Belmont, CA (US)

Correspondence Address: RENATA I. DUMITRASCU, 101 MAPLE STREET APT 2202, REDWOOD CITY, CA, 94063, US

(21) Appl. No.: 10/173,318

(22) Filed: Jun. 17, 2002

(51) Int. Cl. A61F 13/06

(52) U.S. Cl. 128/893

(57) ABSTRACT

An article of skin protection for the foot comprising a sheet of colored or transparent, thin, flexible material having sufficient size to wrap around a portion of the skin area of the metatarsal of the foot and toes leaving the toenails exposed. The underside of article has means of adhering to skin. Article cut out in a shape whereby, when applied, each toe is partially covered by extension of said sheet. The article also covers the area where typically part of open-toed footwear touches the skin of the dorsum. When applied, said article protects skin for damage, while being nearly invisible. Acting as a barrier between footwear and other elements and skin, said article will increase foot health and protect foot appearance. Said article can be manufactured from materials available on the market. Its outline can be achieved through a simple cutting procedure on a sheet of material.

### REFERENCES CITED

U.S. PATENT DOCUMENTS

| | | | |
|---|---|---|---|
| 5683354 | November, 1997 | Levy | 602/54 |
| 6159173 | December, 2000 | Morales | 602/30 |

### BACKGROUND OF THE INVENTION

1) Field of Invention

This invention relates to skin protection adhesive patches, specifically to an improved protection for the upper side of feet and toes.

2) Description of the Prior Art

Wraps, footwear inserts or skin adhesive wound bandages have been used to protect the foot skin. Though adhesive bandages of different shapes and sizes are commonplace, no single one-piece article covers each toe at the same time as well as the forefoot. Also, none are designed to be used both for prevention, on healthy skin, as well as for protection of damaged skin. In the prior art, adhesive bandages are used to treat wounds, while wraps and inserts are used to prevent damage from occurring to the skin of the foot. The advantage of my invention is that the same article can serve all those functions at the same time.

Consider U.S. Pat. No. 5,683,354 to Levy, entitled ADHESIVE BANDAGE FOR A DIGIT OF A HUMAN HAND OR FOOT, which has the drawback that it covers only one digit at a time. Though this may work for one or two damaged digits, it is time consuming and tedious to use daily for protection of healthy skin on all toes. The advantage of my invention is that it protects all toes at the same time with one single article.

Consider also U.S. Pat. No. 6,159,173 to Morales, entitled SPORTS FOREFOOT JOINT STABILIZER, which also does not wrap around each toe and is not adhesive. The wrap is also too visible and cosmetically unappealing to wear under dressy footwear. The advantage of my invention is that it covers all toes, the skin of the forefoot, adheres to the skin, and is cosmetically appealing.

3) Disclosure Statement

I am not aware of any transparent or skin-colored skin adhering device specifically designed for protection of healthy as well as damaged skin on the upper foot and toes that can be worn with any footwear, no matter how revealing. My invention would allow for cosmetically appealing protection of the most visible damaged areas of the forefoot. This type of damage is typically caused by footwear worn directly on the skin, where the footwear is rubbing against the skin. I such instances, sweat, dirt and other fluids can create wounds, discomfort, pain, slipping and falling, limiting movements and creating unattractive scarring of the foot. All these problems can be safely prevented, eliminated or minimized by my invention.

### SUMMARY OF THE INVENTION

This invention relates to skin protection adhesive patches, specifically to an improved protection for the upper side of feet and toes.

The common problem of preventing tears and cuts in the skin of the foot caused by footwear has been thus far addressed in two ways: by making the portion of the shoe coming into contact with the skin softer and more pliable, or by the addition of various forms of cushioning inserts. Another branch of products exists which seeks to treat the damage after it occurs. These products typically adhere to the skin around the damaged area and prevent direct contact by footwear with the wound.

Given the wide variety of footwear and the frequency with which people acquire and discard footwear, damage to the skin has become an inevitable occurrence, particularly in footwear that is worn directly on the skin such as sandals, mules and slippers. This damage is most frequent in areas of the world where the climate compels people to wear footwear directly on the skin. This type of damage is also frequent in wearers of high-heeled footwear that tends to push this portion of the foot against the footwear

Accordingly, several objects and advantages of my invention are:

c(1) The ability to prevent damage to the area of the s most frequently damaged in any type of footwear, especially open toed footwear such as sandals, mules and slippers through a skin-adhering ergonomically shaped device that is nearly invisible,

(2) Said device does not require alteration of the footwear, and will not distort the footwear in any way,

(3) Said device will not fall off during normal movement and wear,

(4) Said device can be disposable after single use,

(5) Said device increases foot health by eliminating the usual skin damages and deformities,

(6) Said device increases walking and moving safety by providing a non-slip layer between foot and footwear unaffected by perspiration, dirt or other fluids, thus increasing range of movement,

(7) By wearing said device, people with sensitive skin on the toes and upper side of foot will no longer have to avoid wearing footwear with straps or footwear that typically damages that area of their skin easily,

(8) Said device can be manufactured out of currently available materials, from one flat sheet of material through a single cutting procedure, thus being easy to manufacture,

(9) Said device increases the cosmetic aspect of bare feet, by protecting them from scarring, blistering, cutting, infections and discoloration,

(10) Said device increases the range of footwear types that can be worn safely,

(11) Said device can be worn for long periods of time,

(12) Said device has an ergonomic shape that complements the shape of the user's forefoot,

(13) Said device does not restrict the movements of the body.

Further aspects and advantages of this invention will become apparent from a consideration of the drawings and ensuing description.

### BRIEF DESCRIPTION OF THE DRAWING

FIG. 1 is a perspective view of the skin adhesive patch.

FIG. 2 is top view of the patch as worn on foot.

FIG. 3 is a view of the patch as worn on feet under sandal-type, high-heeled footwear.

FIG. 4 is a view of the sole of the foot wearing the patch.

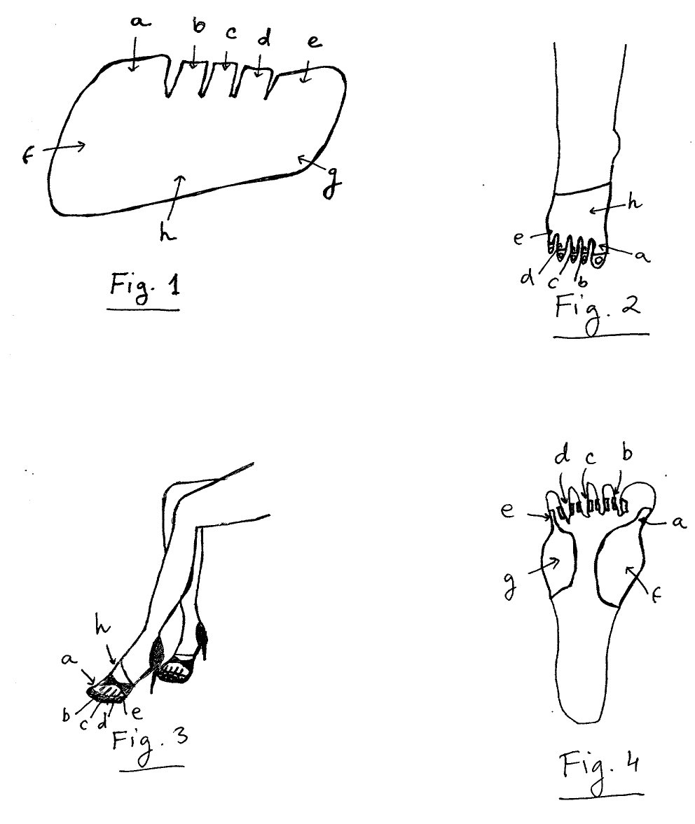

### DESCRIPTION OF THE PREFERRED EMBODIMENT

Before explaining the present invention in detail it is to be understood that the invention is not limited in its application to the details of construction and arrangement of parts illustrated in the accompanying drawing, since the invention is capable of other embodiments and of being practiced or carried out in various ways. Also, it is to be understood that the phraseology or terminology employed therein is for the purpose of description and not limitation.

The preferred embodiment for the invention disclosed is in FIGS. 14 of the drawing, in which like reference letters refer to like and corresponding parts throughout the several views. I will heretofore refer to the invention as the patch for ease of communication.

The patch consists of a piece of flexible, transparent or colored material that can be applied to the skin by means of adhesion. Examples of materials that could be used could be of the type manufactured by 3M (3M Center, P.O. Box 33800, St. Paul Minn. 55133-3800) under the brand name Tegaderm™ or that manufactured by Johnson & Johnson (One Johnson & Johnson Plaza, New Brunswick, N. J. 08933) under the brand name Bioclusive™. These are just examples and in no way limit the type of material that can be used to manufacture the patch. The patch can be manufactured from any flexible material that can safely be adhered to skin.

The patch has two sides: one that will adhere to the foot, and one that will come in contact with the footwear. The side that comes in contact with the footwear can be smooth, or textured, or a combination of both in order to maintain a non-slip surface. In the drawing, (a) wraps around the big toe, (b), (c) and (d) each wrap around a toe, and (e) wraps around the little toe. (f) and (g) wrap around the sole of the foot and can have a particularly non-slip surface to increase wearing safety. (f) and (g) can keep the foot from moving and slipping against the insole of the footwear. (h) is the portion that comes into contact with the top of the footwear and can protect the softest skin area of the foot from a main footwear pressure point. All portions of patch, (a), (b), (c), (d), (e), (f), and (g) can have reinforced padding to further protect the foot. They can also be applied on top of other foot adhesion bandages, pads or other treatments.

The operation of my invention is as follows: (1) Place patch on top of foot and toes in a position most comfortable and covering the skin areas where protection is desired. (2) Press firmly on every portion of patch, making sure the edges are making full contact with the skin. (3) Wear footwear over foot as normal. (4) When desired, peel off patch.

The patch will enable people who wear footwear against bare skin to have a barrier of protection for their skin. High heels wearers will especially benefit because the portion of the foot skin covered by the patch is most stressed and damaged by that type of footwear. People concerned with foot appearance will find the patch to be a tool in their beauty regiment. The patch can be worn every day over healthy or torn skin.

Although but a single embodiment of the invention has been disclosed and described therein, it is obvious that many changes can be made in the size, shape, arrangements, color and detail of the various elements of the invention without departing from the scope of the novel concepts of the present invention. More specifically, it should be understood that the present invention adhesive patch may include any length or width, thickness, curvature or shape of each toe portion, as well as any length, width, thickness, curvature or shape of portion covering metatarsal area. All such variations and modifications are intended to be included within the scope of this invention as defined by the claims.

What is claimed is:

1. An article of protection for the healthy or damaged skin of the forefoot comprising of a piece of flexible colored or transparent material with means of adhesion; the shape of said article can be flat or ergonomically molded, permitting it to wrap around all or a portion of the metatarsal area of the foot and around each toe separately, leaving the toenails exposed; said article extending over portion of the dorsal foot skin usually coming in contact with a slipper, mule or sandal-type footwear, covering all or part of the metatarsal area,

2. The article recited in claim 1 wherein padding material or dressing elements are used to reinforce the article in its entirety, or partially, in areas where the typical open toed type footwear makes contact with the forefoot skin covered by said article,

3. The article recited in claim 1 wherein texture is used to create a non-slip surface on the top surface of article in its entirety, or partially, in areas where the typical open toed type footwear makes contact with the forefoot covered by said article.
